(19) United States
(12) Patent Application Publication
(10) Pub. No.: US 2022/0036977 A1
(43) Pub. Date: Feb. 3, 2022

(54) SYSTEM AND METHOD FOR ANALYZING AND PROVIDING PERSONALIZED COSMETIC INFORMATION

(71) Applicant: LOGSYNK CO., LTD., Seoul (KR)

(72) Inventor: Soo Il LEE, Cheonan-si, Chungcheongnam-do (KR)

(73) Assignee: LOGSYNK CO., LTD., Seoul (KR)

(21) Appl. No.: 17/290,988

(22) PCT Filed: Nov. 19, 2019
(86) PCT No.: PCT/KR2019/015806

§ 371 Date: May 3, 2021

(30) Foreign Application Priority Data
Dec. 27, 2018 (KR) 10-2018-0170377

(51) Int. Cl. G16H 10/20; A61B 5/00; G16H 10/40; G16H 30/40; G16H 10/60

(52) U.S. Cl. CPC G16H 10/20; A61B 5/443; A61B 5/442; A61B 5/4866; A61B 5/0022; A61B 2576/02; A61B 5/0077; G16H 10/40; G16H 30/40; G16H 10/60; A61B 5/0013

(57) ABSTRACT

A system and method for providing personalized cosmetic information objectively and scientifically present personalized cosmetics suitable for an individual's skin type by a comparative analysis between genetic skin test result data, from which an individual's skin condition can be scientifically detected, and data obtained by a phenomenological self-skin examination and a skin condition measurement instrument.

### TECHNICAL FIELD

The present invention relates to a system and method for analyzing and providing personalized cosmetic information. More specifically, the present invention is to objectively and scientifically present personalized cosmetics suitable for an individual's skin type by a comparative analysis between skin genetic test result data, from which an individual's skin condition can be scientifically detected, and data obtained by a phenomenological self-skin examination and a skin measurement instrument.

### BACKGROUND ART

Each individual has different skin conditions. Particularly, people even in the same age and with the same skin type may have greatly different skin conditions depending on the inner and outer environments of the individual. As science develops, the skin is severely affected by exposure to various contaminated environments, exposure to UV light due to depletion of the ozone layer, many types of stress, etc. Thereby, the function of the skin deteriorates and serious cases are on a rise where skin problems occur.

Recently, functional cosmetics for maintaining and recovering healthy skin conditions are sold in the cosmetic field. Functional cosmetics have functions for keeping the skin conditions healthy as well as treating the skin, and accordingly there are many different types of functional cosmetics, and functional cosmetics have been gradually specialized. Consumers also tend to prefer functional cosmetics capable of keeping the skin healthier to conventional cosmetics with simple makeup function.

Now, however, each user merely recognizes his/her skin condition through simple skin examination questionnaires and simple skin testing. Thus, it is difficult to completely rely on information of the skin condition.

As a conventional method for providing personalized cosmetics by measuring skin condition, Korean Patent No. 10-0371861 discloses a novel system for analyzing the skin condition capable of obtaining skin images remotely by using a skin image obtaining device and a skin condition analyzing server accessed through a data communication network and analyzing the skin condition, thereby selecting suitable cosmetics as a result thereof.

Conventional techniques as described above are not based on genetic testing results, from which an individual's skin condition can be scientifically detected, and thus it is difficult to provide scientific cosmetics suitable for an individual's skin condition based on accurate data. Further, such techniques examine the skin condition through simple analysis which compares skin information obtained from the skin images and reference skin information data. Accordingly, said techniques cannot provide reliable cosmetics because they cannot analyze subsidized skin conditions caused by exposure to various contaminated environments, exposure to UV light due to depletion of the ozone layer, many types of stresses, etc. and thus analysis risks are generated from errors in the skin information data.

According to ‘a method and system for providing cosmetic information using skin genetic information’ disclosed in Korean Patent No. 10-0874825 which was invented in order to solve these problems, the patent reference discloses a method for providing personalized cosmetics, comprising analyzing an individual's skin condition through objective and scientific skin genetic tests, precisely qualifying and quantifying expression levels of specific genes related to each user's skin moisturizing, elasticity, wrinkle and whitening so as to improve the skin condition based on the results thereof, and then adding functional components capable of more effectively improving the skin condition based on the results thereof.

Said prior art reference relates to a technique of analyzing gene expression levels of a subject, comparing the same with a constructed reference database to determine a grade of the current skin condition of the subject, and then providing cosmetics having functional components and contents. Thus, said technique, which may be based on accurate scientific data analysis, determines a grade of the current skin condition of the subject by analyzing gene expression levels of the subject, not by performing a comparative analysis between the current skin condition of an individual and the genetic analysis data. As such, the time of analyzing genes is different from the time of the present subdivided skin conditions caused by exposure to various contaminated environments, exposure to UV light due to depletion of the ozone layer, many types of stress, etc. Thus, the current accurate skin condition of an individual cannot be confirmed, and accordingly reliable personalized cosmetics cannot be provided. The questionnaire data of an individual suggested by the prior art reference simply compares each individual's own opinion and genetic information data, which makes it difficult to precisely analyze the current skin condition of an individual. Therefore, the prior art reference cannot provide scientific and reliable personalized cosmetics.

As to systems that have recently been developed, in the case of measuring the skin condition to examine the current skin condition, there are programs, etc., which identify the wrinkle condition and simply digitize a change in skin color or physical conditions such as elasticity, etc., using an image analysis system. However, there are errors in evaluation of change in the skin, and thus it is difficult to perform objective and accurate evaluation. Further, considering that the skin condition varies due to problems of inner parts of the body, merely providing ready-made cosmetic products applied to the skin available in the market has many limits in maintaining and improving healthy skin conditions in response to various changes and phenomenon occurring to the skin.

Accordingly, there are demands for personalized cosmetic information capable of increasing customers' satisfaction and reliability by providing scientific and objective data to provide personalized cosmetics.

### DETAILED DESCRIPTION OF INVENTION

### Technical Task

It is an object of the present invention to analyze an individual's skin condition using a scientific method with innate and acquired skin biometric information results and suggest personalized cosmetics for maintaining and improving skin conditions and physical health and satisfying customer's own needs.

Specifically, the present invention has a technical task of providing a means for providing personalized cosmetics suitable for the current skin symptoms of a subject by comparing skin genetic testing results, from which an individual's skin condition can be correctly detected, and a currently examined skin condition.

### Method for Solving the Technical Task

The present invention performs a comparative analysis of each subdivided item between subject's genetic percentage data, percentage data of current skin conditions obtained by a skin measurement terminal and percentage data of self-examination items, produces a 2-English letter code for the subject's current skin conditions to determine the code as an examination code for examining skin problems, and provides personalized cosmetics suitable for the determined examination code.

### Effect of the Invention

The present invention has an effect of increasing customers' satisfaction and reliability by providing more scientific and objective data on customers' skin conditions thereby providing personalized cosmetics for each individual.

The present invention has an effect of comparing each subdivided item between data on precisely qualified and quantified expression levels of genes related to each individual's skin and data on self-examination questionnaire items to determine a subdivided examination code according to skin symptoms, thereby providing convenience in prescribing cosmetics and also providing correct and scientific personalized cosmetics according to the examination code.

### BRIEF DESCRIPTION OF DRAWINGS

FIG. 1 is a block diagram of the whole system circuit according to the present invention;

FIG. 1a is a classification table of 28 genetic items related to skin, vitamin, obesity and hair loss;

FIG. 1b is a classification table of a list of 33 skin and metabolism self-examination questionnaire items according to the present invention;

FIG. 1c is a genetic analysis skin examination reference table according to the present invention;

FIG. 1d is a skin symptoms examination reference table according to the present invention;

FIG. 1e is a view illustrating an example of 2-letter examination codes and an examination algorithm;

FIG. 1f is a view illustrating correlated items between skin-related subdivided items, metabolic functions of human body such as detox, metabolism and damage recovery, and genetic items and self-examination items corresponding thereto according to the present invention;

FIG. 1g is a view illustrating correlated items between skin-related subdivided items, metabolic functions of human body such as detox, metabolism and damage recovery, genetic items and self-examination items corresponding thereto, and correlation regarding recommendation of health functional food according to the present invention;

FIG. 2 is a flowchart for explaining the method according to the present invention; and

FIG. 3 is a view illustrating a skin measurement algorithm of the IoT skin measurement terminal according to the present invention.

### BEST MODE FOR CARRYING OUT THE INVENTION

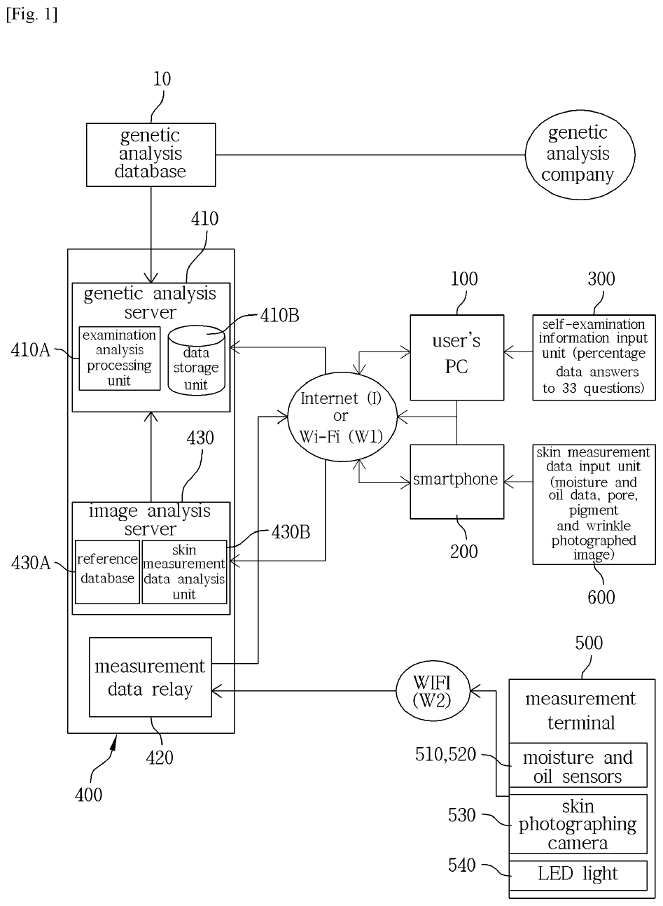

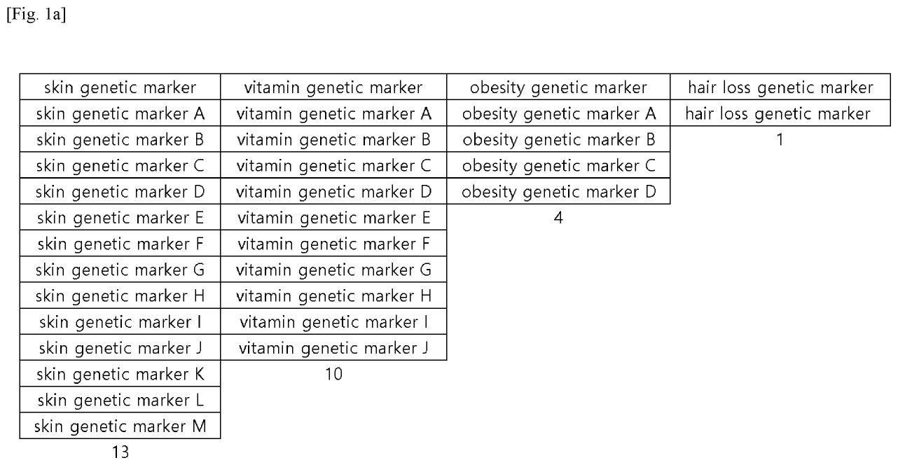

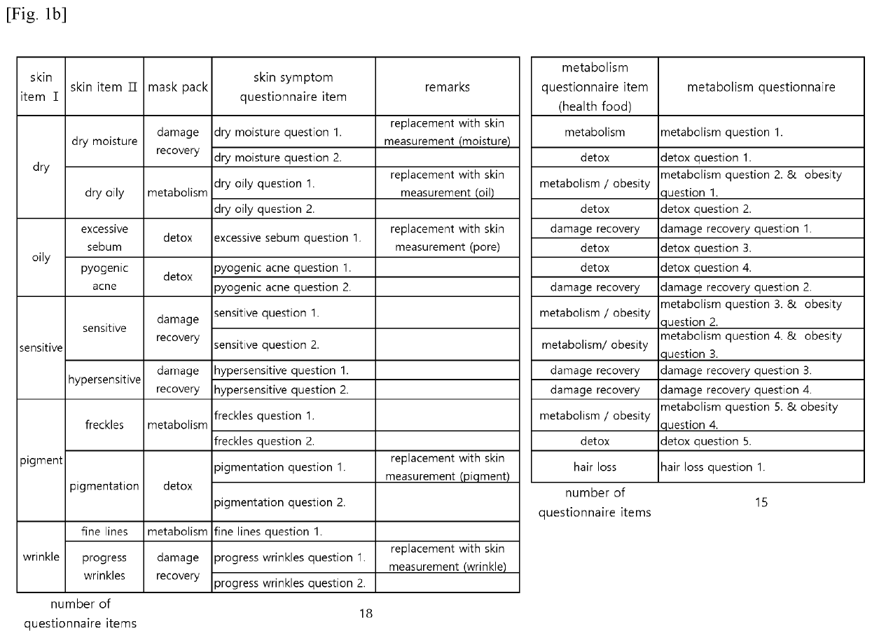

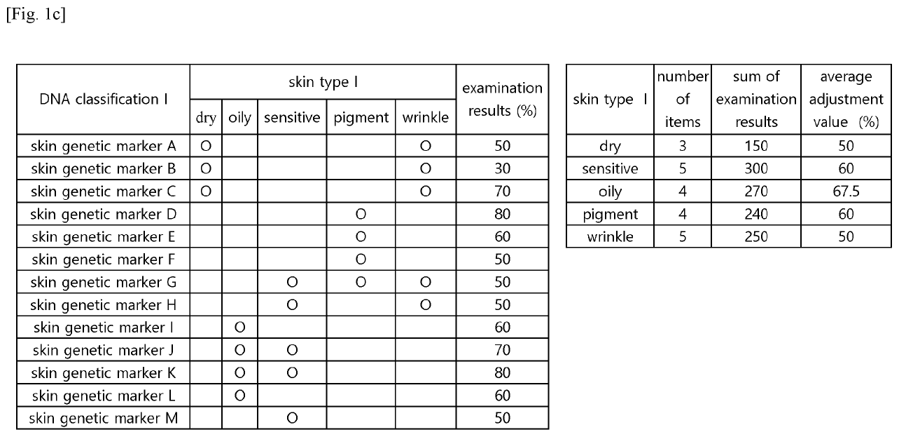

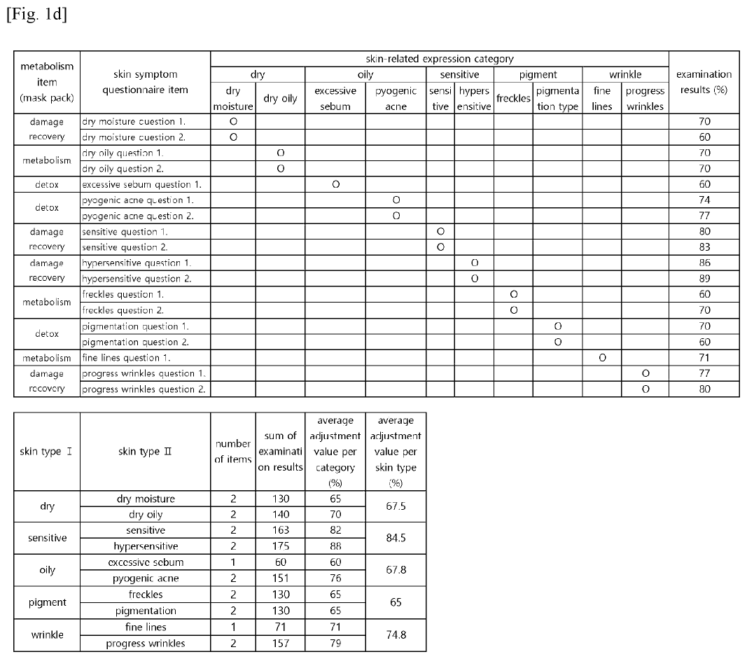

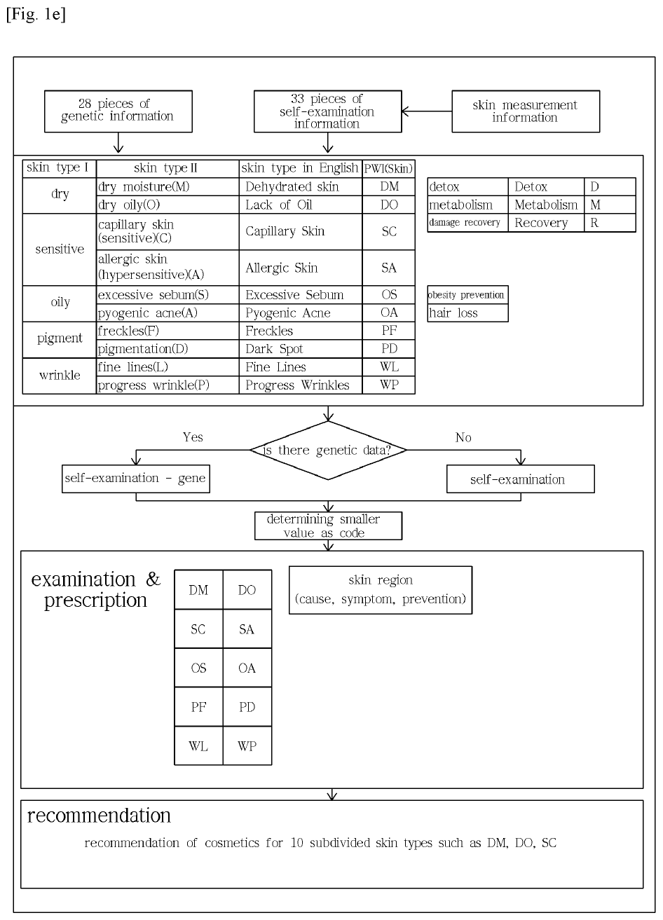

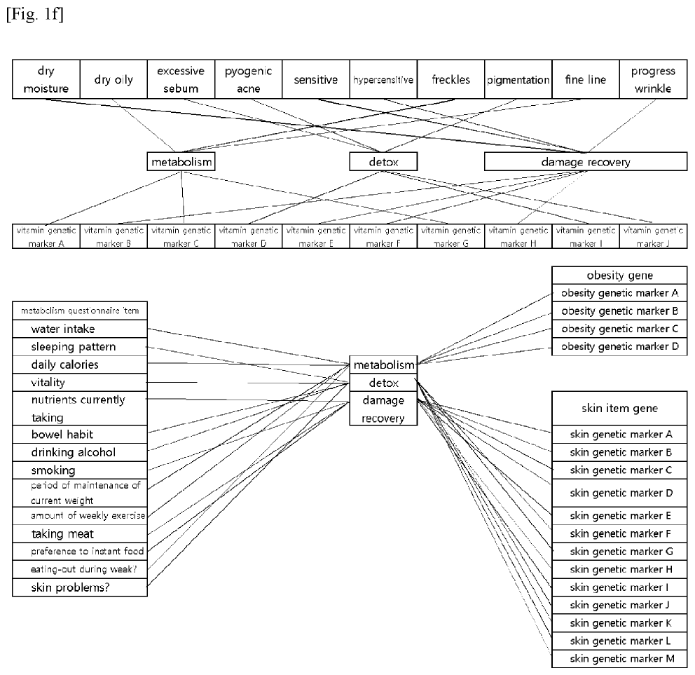

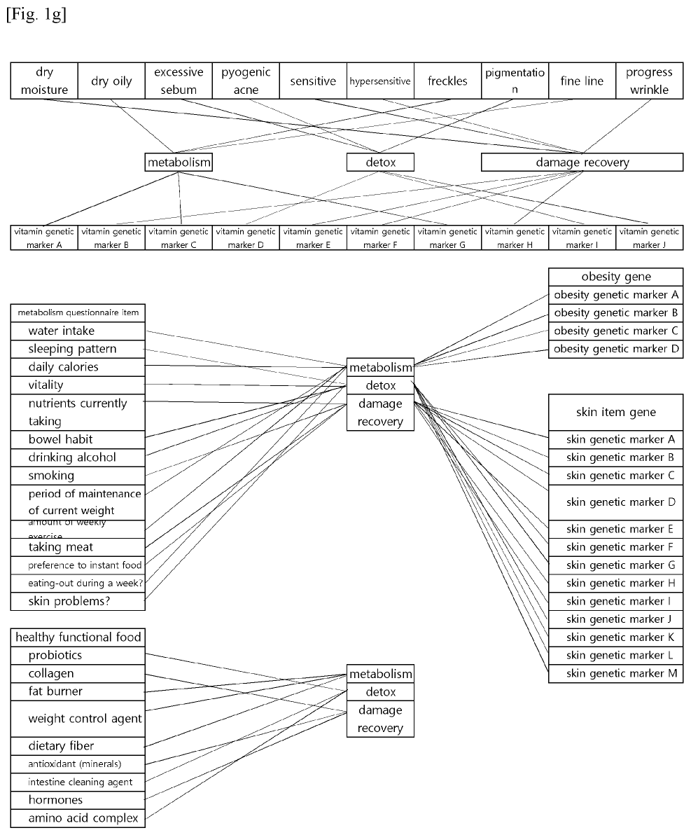

FIG. 1 is a block diagram of the whole system circuit according to the present invention.

The present invention comprises a percentage database (10) for 28 gene items related to skin, vitamin, obesity and hair loss genes received from a genetic analysis company, as illustrated in FIG. 1a.

Also, the present invention comprises a self-examination information input unit (300) for inputting, through Internet (I) or Wi-Fi (Wi), self-examination percentage data obtained by checking, at a user's PC (100) and a smartphone (200), each of 33 questionnaire items in a skin and metabolism self-examination list illustrated in FIG. 1b and converted into percentages using the Likert scale of measurement.

As illustrated in FIG. 1a, genetic testing items are a total of 28 gene items including 13 skin genotype categories of skin genetic marker A, skin genetic marker B, skin genetic marker C, skin genetic marker D, skin genetic marker E, skin genetic marker F, skin genetic marker G, skin genetic marker H, skin genetic marker I, skin genetic marker J, skin genetic marker K, skin genetic marker L, and skin genetic marker M, as skin-related genes, 10 vitamin gene categories of vitamin genetic marker A, vitamin genetic marker B, vitamin genetic marker C, vitamin genetic marker D, vitamin genetic marker E, vitamin genetic marker F, vitamin genetic marker G, vitamin genetic marker H, vitamin genetic marker I, and vitamin genetic marker J, as genetic items forming nutrients and metabolism having a correlation with the skin, 4 obesity gene categories of obesity genetic marker A, obesity genetic marker B, obesity genetic marker C, and obesity genetic marker D, and 1 hair loss gene category.

The 13 skin-related genes in the 28 gene items are classified into 5 large-category skin conditions, dry, sensitive, oily, pigment and wrinkle, as shown in FIG. 1c (genetic analysis skin examination reference table), wherein the dry condition is subdivided into skin genetic marker A, skin genetic marker B and skin genetic marker C, the oily condition is subdivided into skin genetic marker I, skin genetic marker J, skin genetic marker K and skin genetic marker L, and the other sensitive, pigment and wrinkle conditions are also subdivided into corresponding genetic markers marked with “O” in the corresponding boxes in FIG. 1c, so as to increase accuracy of measurement of an individual's skin condition for skin-related genes.

Also, the list of 33 skin and metabolism self-examination items suggested in FIG. 1b is designed to classify the skin conditions into large-category skin conditions and subdivided skin conditions to extract percentage data for each item corresponding to cosmetics. Specifically, as to the contents of skin examination questionnaire, one or more questions are set for each large category. That is, the questions are subdivided as follows: dry (dry moisture: dry moisture questions 1, 2, dry oily: dry oily questions 1, 2), oily (excessive sebum: excessive sebum question 1, pyogenic acne: pyogenic acne questions 1, 2), sensitive (sensitive: sensitive questions 1, 2, hypersensitive: hypersensitive questions 1, 2), pigment (freckles: freckles questions 1, 2, pigmentation: pigmentation questions 1, 2), and wrinkle (fine line: fine line questions 1, 2, progress wrinkles: progress wrinkles questions 1, 2). Such subdivided questionnaire items can be a basis on which each individual's skin condition can be precisely analyzed. As illustrated in FIG. 1b, such subdivided questionnaire items can be used for suggesting metabolism questionnaire items for recommending mask packs (metabolism questionnaire items including damage recovery, metabolism and detox corresponding to each of dry, oily, sensitive, pigment and wrinkle) and health functional food, in addition to recommendation of cosmetics described below. As illustrated in FIG. 1f and FIG. 1g, the skin-related subdivided items according to the present invention may be associated with detox, metabolism and damage recovery, which are body metabolism functions, correlated items of gene items and self-examination items corresponding thereto, and recommendation of health functional food.

In order to provide satisfactory and reliable cosmetics suitable for an individual's skin condition, the present invention classifies skin conditions into 5 large-category skin conditions, dry, sensitive, oily, pigment and wrinkle, as illustrated in FIG. 1d (skin symptoms examination reference table), and the 5 large-category skin conditions are divided into 10 subdivided skin conditions, dry (dry moisture/dry oily), sensitive (sensitive/hypersensitive), oily (excessive sebum/pyogenic acne), pigment (freckles/pigmentation) and wrinkle (fine line/progress wrinkles). Percentage data obtained therefrom is stored in a data storage unit (410B) in a genetic analysis server (410) in a cloud server (400), wherein for the 10 subdivided items, respective examination codes thereof are produced by an examination analysis processing unit (410A) as described below. The produced subdivided examination codes allow easy and convenient selection of personalized cosmetics. In the case of self-examination as illustrated in FIG. 1d, skin symptoms are examined by checking one or more questions suggested for each of the 10 subdivided items. For example, in the case of dry moisture type, dry moisture question 1 and dry moisture question 2 are suggested for checking.

That is, subdivided examination codes are produced by combining a combined 2-English letter (DM, DO, SC, SA, OS, OA, PF, PD, WL, WP) for each of the 10 skin classified items of dry moisture (Dry Moisture), dry oily (Dry Oily), sensitive (Sensitive Capillary), hypersensitive (Sensitive Allergy), excessive sebum (Oily excessive Sebum), pyogenic acne (Oily Acne), freckle (Pigment Freckles), pigmentation (Pigmentation Type), fine line (Wrinkle fine Line) and progress wrinkle (Wrinkle Progress wrinkles) and a capital letter (D, M, R) for 3 metabolism items of detox, metabolism and recovery, to be classified into an acronym PWI code of personalized wellness solution. A percentage data value with a smallest value obtained by the examination analysis processing unit (410A) is output to a combined 2 English-letter examination code, thereby providing a personalized cosmetic product corresponding to the examination code as a solution.

Specifically, skin types are primarily classified into large-category skin type I (D, S, O, P, W) of dry, sensitive, oily, pigment and wrinkle, and these items are further classified into subdivided skin type II (M, O, C, A, S, A, F, D, L, P). The first English letters of the large-category skin type I and subdivided skin type II make 2-letter examination codes (DM, DO, SC, SA, OS, OA, PF, PD, WL, WP) for the skin. These 2-letter codes form the front 2 letters of a Personalized Wellness Index (PWI) code with 3 letters.

An example of the aforementioned 2-letter examination codes is illustrated in FIG. 1e.

Also, the present invention comprises a data storage unit (410B) constituting a genetic analysis server (410) in a cloud server (400) for storing the genetic percentage data of the genetic analysis database (10) and the self-examination percentage data obtained by the self-examination information input unit (300), and an examination analysis processing unit (410A) for a comparative analysis of the stored input data.

Also, the present invention may comprise a data relay (420) in the cloud server (400) for inputting, through Wi-Fi (W2), moisture and oil data and pigment, pore and wrinkle image data obtained by an IoT skin measurement terminal (500) and relaying the data to a skin measurement data input unit (600).

This is to identify individual's skin and body characteristics through genetic testing items of FIG. 1a for innate factors, confirm external factors such as age, climate, season, etc. using questionnaires that can be examined by an individual as illustrated in FIG. 1b, and identify parts that cannot be examined by an individual by photographing the skin surface using a skin photographing camera (530) constituting the IoT skin measurement terminal (500), in order to scientifically approach an individual's skin characteristics and more precisely examine the skin conditions. According to the present invention, the moisture and oil data and pigment, pore and wrinkle image data that cannot be obtained from the self-examination of FIG. 1b are replaced with data measured by the IoT skin measurement terminal (500) to correctly measure the current skin condition of an individual.

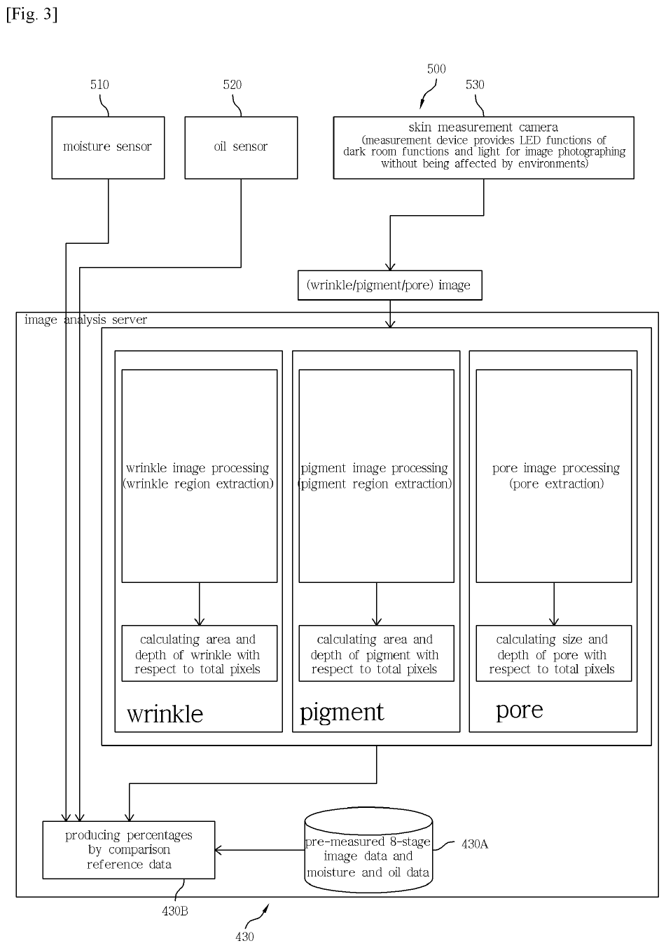

Further, as illustrated in FIG. 1 and FIG. 3 (skin measurement algorithm), the IoT skin measurement terminal (500) comprises moisture and oil sensing units (510)(520), a skin photographing camera (530) and an LED light unit (540), wherein the data measured by the moisture and oil sensing units (510)(520) and skin photographing camera (530) is compared with reference data of a reference database (430A) having 8-stage image data in an image analysis server (430) and converted into percentage data by a skin measurement data analysis unit (430B).

The skin photographing is made using the skin photographing camera (530). Conventional skin measurement is performed by installing a skin examination application, connecting an IoT device to the application one to one using a Wi-Fi network, and transmitting images photographed by the IoT device to the application, to confirm numerical values, etc., with data registered in a system stored in the application. By comparison, according to the present invention, the skin photographing by the skin photographing camera (530) is made by connecting the IoT skin measurement terminal (500) to the web-based skin examination cloud server (400) using the data relay (420) with relaying functions in the cloud server (400), thereby receiving a solution.

Particularly, the skin measurement data input unit (600) selects the serial number of the IoT skin measurement terminal (500) which is registered in the cloud server (400) and identifies the serial number to keep a connection between the skin measurement data input unit (600), measurement data relay (420) and skin measurement terminal (500), and the images photographed by the IoT skin measurement terminal (500) are allowed to be checked at the user's PC (100) and smartphone (200).

The IoT skin measurement terminal (500) having the aforementioned constitution can provide a web-based examination solution which exchanges a protocol with the cloud server (400) and the user's PC (100) and smartphone (200), thereby allowing a connection between the IoT skin measurement terminal (500) and cloud server (400) even using different networks, without using the same Wi-Fi network. When the smartphone (200) accesses the cloud server (400), a web-based examination solution, it can be used to be connected with the IoT skin measurement terminal (500) through tethering (technique capable of allowing other devices to access the Internet using a device already connected to the Internet, such as smartphone).

For the current skin conditions, moisture, oil, wrinkle, pigment and pore are measured by the IoT skin measurement terminal (500) having the aforementioned constitution, and a specific embodiment is described with reference to FIG. 3 as follows.

Moisture and oil items of an individual may be measured by the moisture and oil sensors (510)(520), respectively, and compared and analyzed with the reference database (430A) to produce percentage values by the skin measurement analysis unit (430B). For wrinkle, pigment and pore images, the IoT skin measurement terminal (500) may have an LED light unit (540) for dark room function and light installed in order to photograph images while not being affected by photographing environments, change modes according to the corresponding items, and photograph each of wrinkle, pigment and pore images at the guided skin site.

The photographed wrinkle image may be subjected to wrinkle image processing (wrinkle region extraction) to calculate the depth and area of the wrinkle with respect to the total pixels, and the 8-stage image data of the reference database (430A) may be compared with the measured data to produce a percentage value.

Further, the pigment image may be subjected to pigment image processing (pigment region extraction) to calculate the depth and area of the pigment with respect to the total pixels, and the 8-stage image data stored in the reference database (430A) may be compared with the measured data to produce a percentage value.

Moreover, the pore image may be subjected to pore image processing (pore extraction) to calculate the depth and area of the pore with respect to the total pixels, and the 8-stage image data of the reference database (430A) may be compared with the measured data by the skin measurement data analysis unit (430B) to produce a percentage value.

In addition, the moisture and oil data and pore, pigment and wrinkle photographed image measurement data measured by the IoT skin measurement terminal (500) may be transmitted to the user's PC (100) or smartphone (200) through the Internet (I) or Wi-Fi (W1) via the data relay (420) in the cloud server (400). The data may be transmitted to the image analysis server (430) by the skin measurement data input unit (600) through the user's PC (100) or smartphone (200). Here, the authentication number of the corresponding IoT skin measurement terminal (500) may be confirmed for authentication processing, and once authentication is confirmed, the skin measurement data of the IoT skin measurement terminal (500) may be transmitted to the image analysis server (430) in the cloud server (400) by operation of the user's PC (100) and smartphone (200). The reference database (430A) constituting the image analysis server (430) and the input skin measurement data may be compared by the skin measurement data analysis unit (430B). The produced percentage data of each of oil, pigment, pore and wrinkle may be transmitted to the genetic analysis server (410).

This is to analyze the percentage data using the image analysis server (430) and transmit the data to the genetic analysis server (410), in order to primarily more reliably analyze the percentage data measured by the IoT skin measurement terminal (500). Since the currently measured data is more important and reliable than self-examination, the moisture and oil data and pore, pigment and wrinkle images, which are included in the self-examination items, are replaced with the percentage data measured by the IoT skin measurement terminal (500) for analysis. For example, in the case of inputting the data obtained by the IoT skin measurement terminal (500) to the genetic analysis server (410) through the image analysis server (430), the dry moisture and dry oily subdivided items of the dry item in the self-examination are replaced with the percentage data measured by the moisture and oil sensors (510)(520) of the IoT skin measurement terminal (500), the excessive sebum and pyogenic acne subdivided items of the oily item in the self-examination are replaced with the pore image photographed by the skin photographing camera (530), the freckles and pigmentation subdivided items of the pigment item in the self-examination are replaced with the pigment image photographed by the skin photographing camera (530), and the fine line and progress wrinkles subdivided items of the wrinkle item in the self-examination are replaced with the wrinkle image photographed by the skin photographing camera (530), so as to be analyzed.

In addition, in the case of inputting only self-examination percentage data without inputting genetic percentage data, the smallest percentage data of the self-examination percentage data may be determined as the corresponding recommendation code.

The genetic analysis server (410) may compare genetic percentage data obtained by the genetic analysis database (10), self-examination percentage data obtained by the self-examination information input unit (300) and skin measurement percentage data obtained by the image analysis server (430). When the self-examination percentage data obtained by the self-examination information input unit (300) and the genetic percentage data obtained by the genetic analysis database (10) are input, an average percentage data value of each genetic item may be subtracted from an average percentage data value of each self-examination item to determine a smallest percentage data value of the percentage data values of the corresponding items as a corresponding recommendation code. When self-examination percentage data and skin measurement percentage data obtained by the image analysis server (430) are input together with genetic percentage data to the analysis server (410), the percentage data of the self-examination item may be replaced with the percentage data of the corresponding skin measurement item, and an average percentage data value of each genetic item may be subtracted from an average percentage data value of each corresponding self-examination item to determine a smallest percentage data value of the percentage data values of the corresponding items as a corresponding examination code, thereby providing a prescription for an individual's skin symptom.

That is, the summed-up percentages of gene items corresponding to dry in the genetic item percentage data input to the genetic analysis server (410) are converted into an average value at the examination analysis processing unit (410A). The summed-up percentages of dry moisture item corresponding to dry of the skin symptom type obtained by checking the self-examination questionnaire item at the user's PC or smartphone and converted using the Likert scale are converted into an average value at the examination analysis processing unit (410A) and at the same time the summed-up percentages of dry oily item corresponding to dry obtained by checking the self-examination questionnaire item and converted using the Likert scale are converted into an average value at the examination analysis processing unit (410A). Dry oily type percentage data is obtained by subtracting the genetic analysis average value corresponding to dry from the dry moisture average value by the self-examination (self-examination analysis average value−genetic analysis average value). Then, a smaller value between the dry moisture type percentage data and dry oily type percentage data is produced at the examination analysis processing unit (410A).

In the same manner as above, the percentage data for each of 8 subdivided skin items corresponding to sensitive, oily, pigment and wrinkle skin types is summed up sequentially (or simultaneously) to produce each average value by the examination analysis processing unit (410A), to determine, as an examination code, the item having a smallest value of the 10 items of dry, sensitive, oily, pigment and wrinkle skin types, thereby providing a prescription for an individual's skin symptom.

Specifically, the present invention compares the genetic analysis percentage values which are innate information, and the percentage values of skin measurement information and 33 self-examination information which are acquired information, for each skin type, to determine a value of which difference value is small as an examination code for each type and if no genetic analysis data is provided, to determine a code which has a small percentage value of self-examination as an examination code. In short, the average values (English letter example: A1˜A5) of the sums of percentage data for the skin genetic marker items corresponding to the respective 5 large-category items (dry, sensitive, oily, pigment, wrinkle) are converted at the examination analysis processing unit (410A). The average values (English letter example B1˜B5) of the sums of percentage data of dry moisture type questions 1 and 2 of the subdivided items checked in the self-examination skin-related 5 large categories (dry, sensitive, oily, pigment, wrinkle), and the respective percentage data on moisture, oil, pore, pigment and wrinkle obtained by the IoT skin measurement terminal (500), C1 (English letter example, moisture and oil), C2 (English letter example, pore), C3 (English letter example, pigment), and C4 (English letter example, wrinkle) are output at the examination analysis processing unit (410A). The average values (English letter example: B′1˜B′5) of the sums of percentage data of questions 1 and 2 corresponding to dry oily type in the B1-B5 are processed at the examination analysis processing unit (410A) to determine the average values of the sum of the B1˜B5 values and B′1˜B′5 values as skin type I (English letter example: BB′1˜BB′5) (for example, first letter of the code dry D). After the BB′˜BB′5-A1˜A5 (subtraction) to obtain K1˜K5 (English letter example) (after comparative analysis), a smallest value (example of second letter of the code, dry moisture: M) of each of the small values (B1˜B5) of the subdivided item questions 1 and 2 is selected from the K1˜K5. A smallest value of the 5 items (dry, sensitive, oily, pigment, wrinkle) is selected as skin type II item (2-letter code, for example, DM) to determine the item as an examination code. The present invention will be described with a specific example as follows.

That is, in the case where the percentage data of skin genetic marker A, skin genetic marker B and skin genetic marker C corresponding to dry in the genetic item percentage data illustrated in FIG. 1c input to the genetic analysis server (410) is 50%, 30% and 70%, respectively, the sum of the percentages (150%) of the 3 items is converted into an average value (50%) at the examination analysis processing unit (410A). The sum of the percentages (130%) of the percentage data 70% of dry moisture type question 1 and the percentage data 60% of dry moisture type question 2 obtained by checking the self-examination questionnaire item illustrated in FIG. 1d and converted using the Likert scale is converted into an average value (65%) at the examination analysis processing unit (410A) and at the same time the sum of the percentages (140%) of the percentage data 70% of dry oily type question 1 and the percentage data 70% of dry oily type question 2 obtained by checking the self-examination questionnaire item illustrated in FIG. 1d and converted using the Likert scale is converted into an average value (70%) at the examination analysis processing unit (410A). Then, an average value 67.5% of the sum of the moisture 65% and oily 70% is set as an average value for large category skin type I (dry code D of FIG. 1e). The genetic analysis average value 50% corresponding to dry is subtracted therefrom (self-examination analysis average value 67.5%−genetic analysis average value 50%) to obtain dry percentage data 17.5%. The item with a smaller value between the moisture 65% and oily 70% in the self-examination is determined as an examination code (DM) for the subdivided skin type II (dry moisture code M of FIG. 1e).

This means that although the average value of genetic item percentage data is 50%, the average value of the dry moisture type percentage data changed by season or surrounding environments, etc. falls to 17.5%. Thus, it indicates that it is necessary to provide a cosmetic suitable for the average value of the genetic item percentage data 50% or greater. Accordingly, the subtraction algorithm of self-examination analysis average value−genetic analysis average value according to the present invention is reasonable.

That is, in order to solve the problem that the correct current skin conditions of an individual cannot be confirmed because the time when analyzing genes is different from the time of the present subdivided skin conditions caused by exposure to various contaminated environments, exposure to UV light due to depletion of the ozone layer, many types of stress, etc., and thus reliable personalized cosmetics cannot be provided, an average value is calculated for each skin symptom of the 10 subdivided items, by summing up each item so as not to be focusing on any one skin symptom. The subtraction algorithm which performs subtraction on these average values through self-examination analysis average value (including skin measurement average value by skin measurement terminal)−genetic analysis average value to determine a smallest value of the items as an examination code is to diagnose that the percentage data of the present skin symptoms caused by exposure to various contaminated environments, etc. is worse than the percentage data information obtained from the genetic analysis of an individual.

Further, the moisture dry type percentage data 17.5% calculated at the examination analysis processing unit (410A) corresponds to dry skin as described above. In the case of dry moisture type, the examination analysis processing unit (410A) determines and produces the examination code ‘DM.’

Also, as processed at the examination analysis processing unit (410A) described above, for each of sensitive, oily, pigment and wrinkle, analysis is processed through subtraction of subtracting genetic average percentage (average adjustment value) from self-examination average percentage (adjustment value for skin type). For example, in the case of sensitive and sensitive capillary wherein the examination code is determined as ‘SC’ and the percentage data of the examination code ‘SC’ is 24.5% (the genetic adjustment value is 60% and the skin type adjustment value is 84.5%), in the case of oily and excessive sebum wherein the examination code is determined as ‘OS’ and the percentage data of ‘OS’ is 15% (the genetic adjustment value is 50% and the skin type adjustment value is 65%), in the case of pigment and freckles wherein the examination code is determined as ‘PF’ and the percentage data of ‘PF’ is 5% (the genetic adjustment value is 60% and the skin type adjustment value is 65%), and in the case of wrinkle and fine line wherein the examination code is determined as ‘WL’ and the percentage data of ‘WL’ is 17% (the genetic adjustment value is 60% and the skin type adjustment value is 77%), the examination analysis processing unit (410A) determines a smallest value ‘PF’ 5% of ‘DM’ 17.5%, ‘SC’ 24.5%, ‘OS’ 15%, ‘PF’ 5% and ‘WL’ 17% as a final examination code according to the skin symptoms.

Thus, from the examination code ‘PF’ 5%, it can be understood that as to the skin symptoms of an individual, problems occur due to pigment freckles. Further, the examination code ‘PF’ is transmitted to the user's PC (100) or smartphone (200) to scientifically and objectively suggest cosmetics for preventing or alleviating the symptoms to an individual who has the ‘PF’ symptoms.

When the percentage data measured by the IoT skin measurement terminal (500) is input to the examination analysis processing unit (410A) as described above, moisture and oil data and pore, pigment and wrinkle photographed image data measured by the IoT skin measurement terminal (500) may be replaced with the checked items in the self-examination questionnaire items illustrated in FIG. 1d, to determine an examination code according to the skin symptoms, as described above.

As described above, the present invention is capable of transmitting the corresponding examination code determined by subtracting the genetic percentage data from the self-examination percentage data to the user's PC (100) or smartphone (200) to confirm the examination code, and then prescribing cosmetics suitable for prevention or treatment of skin symptoms according to the determined examination code.

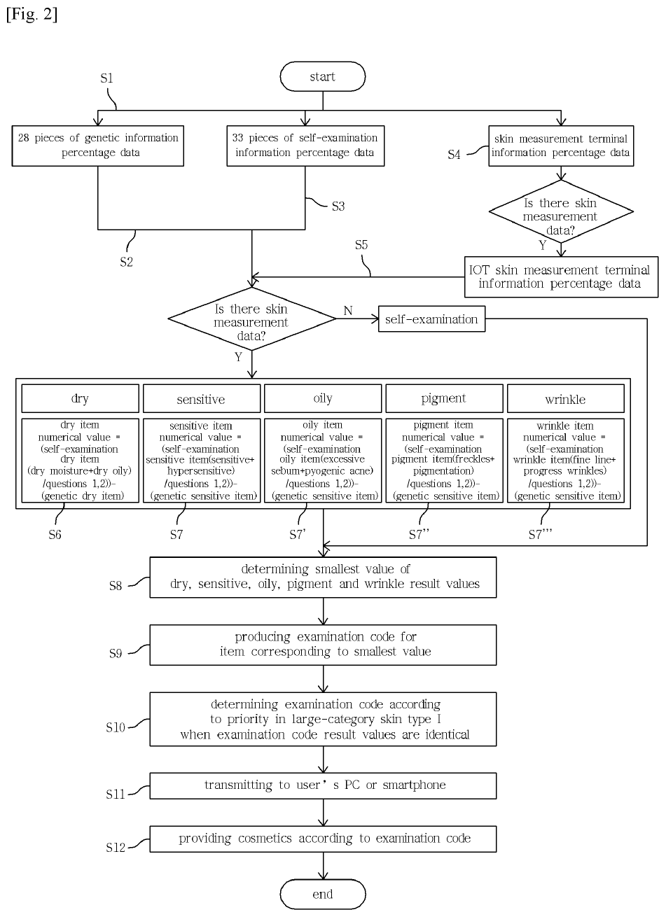

A method for analyzing and providing personalized cosmetic information according to the present invention is described with reference to the flow chart of FIG. 2 based on FIG. 1.
The method comprises: a step (S1) of receiving genetic information which is receiving, from a genetic database, 28 pieces of skin-related percentage data received from a genetic analysis company; a step (S2) of transmitting genetic information which is transmitting the genetic percentage data to a genetic analysis server in a cloud server through HTTP REST API; a step (S3) of transmitting self-examination skin condition information which is transmitting self-examination skin region percentage data obtained from self-examination questionnaire items using the Likert scale to the genetic analysis server in the cloud server through a self-examination information input unit; a step (S4) of measuring skin which is measuring moisture, oil, pore, pigment and wrinkle conditions through a skin measurement algorithm of an IoT skin measurement terminal; a step (S5) of transmitting the skin measurement data of the IoT skin measurement terminal wherein checked items in the self-examination questionnaire items are replaced with moisture and oil data and pore, pigment and wrinkle photographed image data measured by the IoT skin measurement terminal to be input, the skin measurement data is transmitted by a data relay in the cloud server to a user's PC or smartphone through Internet or Wi-Fi to identify an authentication number of the corresponding IoT skin measurement terminal for authentication processing, and once authentication is confirmed, the skin measurement data of the corresponding IoT skin measurement terminal is transmitted to an image analysis server in the cloud server by operation of the user's PC or smartphone to compare, by a skin measurement data analysis unit, a reference database constituting the image analysis server and the skin measurement data input, such that the produced percentage data of each of moisture, oil, pigment, pore and wrinkle is transmitted to the genetic analysis server; a step (S6) wherein the sum of percentage data of skin genetic marker items corresponding to dry in the genetic item percentage data input to the genetic analysis server is converted into an average value at an examination analysis processing unit, the sum of percentages of the percentage data of dry moisture type question 1 and the percentage data of dry moisture type question 2 obtained by checking the self-examination questionnaire item and converted using the Likert scale and the moisture and oil data and pore, pigment and wrinkle photographed image data measured by the IoT skin measurement terminal corresponding thereto are converted into an average value at the examination analysis processing unit, and at the same time the sum of percentages of the percentage data of dry oily type question 1 and the percentage data of dry oily type question 2 obtained by checking the self-examination questionnaire item and converted using the Likert scale is converted into an average value at the examination analysis processing unit, then the average value of the sum of the moisture and oil is set as an average value for large-category skin type I, the genetic analysis average value corresponding to dry is subtracted therefrom (self-examination analysis average value−genetic analysis average value) to obtain dry percentage data, and a smaller value between the moisture and oil items in the self-examination is determined as a dry moisture type examination code of subdivided skin type II; a step (S7)(S7′)(S7″)(S7′″) wherein an average value of percentage data of each genetic item of sensitive, oily, pigment and wrinkle, an average value of percentage of each sensitive, oily, pigment and wrinkle checked in the self-examination items and the moisture and oil data and pore, pigment and wrinkle photographed image data measured by the IoT skin measurement terminal corresponding thereto, which are input to the genetic analysis server of the cloud server, are compared and analyzed at the examination analysis processing unit of the genetic analysis server as in the step (S6) for the subdivided skin type II items (8 items) for each of sensitive, oily, pigment and wrinkle; a step (S8) of determining a smallest value (%) of the subdivided skin type II items (10 items) corresponding to the large-category skin type I (5 items) compared and analyzed in the step of subtraction processing (S6)(S7)˜(S7′″); a step (S9) of analysis processing which is determining the code produced in the step (S8) as an examination code according to a prescription for skin symptoms; a step (S10) of determining in the order of dry<sensitive<oily<pigment<wrinkle when the result values of the examination codes determined in the analysis processing step (S9) are identical; a step (S11) of transmitting the determined examination code to the user's PC or smartphone; and a step (S12) of providing a cosmetic that can solve skin problems based on the determined examination code.

In the step (S10), the reason for having an order of priority for determining the examination code when the result values of the examination codes are identical is that the present invention provides an algorithm taking a small value during the analysis processing. Thus, it can be understood that the skin problem of dry item having a small value in the order affects the skin preferentially to the skin problem of wrinkle item.

In the step (S6), when the result value of dry moisture type and the result value of dry oily type in the dry item are identical to each other, preferably the dry moisture type is determined as the examination code, and when the result value of sensitive and the result value of hypersensitive in the sensitive item are identical to each other, preferably the result value of sensitive is determined as the examination code. Preferably, for oily types having the same values, the result value of excessive sebum is determined as the examination code, for pigment types having the same values, the result value of freckle is determined as the examination code, and for wrinkle types having the same values, the result value of fine line is determined as the examination code.

According to the present invention described herein, the percentage result value of genetic measurement, which is innate characteristics, and the percentage result value of self-examination, which is acquired characteristics, are processed at the examination analysis processing unit to produce the examination code through analysis of difference between the innate and acquired values. The present invention matches relevant items with each other through skin surface photographing in order to compensate an individual's skin phenomenon and a portion the individual recognizes obtained by self-examination (questionnaire) and unify all numerical values to percentages to confirm statistical result values.

The present invention determines 10 skin items according to skin symptoms (values converted into percentages according to genes, self-examination, skin measurement, etc.), classifies dry and oily skin types that occur due to an imbalance between oil and moisture into dry, sensitive, oily, pigment and wrinkle, while combining with the aging process, and subdivides the same into 10 skin items, unlike conventional skin types of dry, oily, combination, normal, etc., to identify an individual's characteristic. When the classified skin type is determined as, for example, dry moisture (DM), the skin type corresponds to dry skin, and the present invention may divide the dry skin into the cause why the corresponding skin type occurs, symptoms thereof and prevention therefor and prescribe personalized cosmetics wherein moisture that is the cause of dry moisture, symptoms of loss of elasticity and complexion purification, and prevention of oil and a regeneration component for the natural moisture layer are reinforced.

For a specific example for description, for example, in the case of the skin corresponding to dry moisture type as a result of the analysis and examination, ‘DM’ is determined as the examination code. The present invention may recommend 5 components including a moisture component for alleviating skin dry symptoms, a component capable of recovering elasticity, a component for purifying complexion, and an oil component and a natural moisture layer regenerating component for preventing dry, in order to solve skin problems corresponding to the ‘DM’ examination code, thereby prescribing personalized cosmetics suitable for the ‘DM’ examination code.

The present invention is explained in detail as above, and the explanation is not intended to limit the scope of the present invention. The explanation is provided for illustrative purposes only, and the scope of the present invention shall be defined by the accompanying claims. It should be construed that simple modifications or alternations within the scope of the claims fall within the scope of the present invention.

What is claimed is:

1. A system for analyzing and providing personalized cosmetic information, comprising:
a genetic database (10) for processing percentage data on 28 genes related to skin, vitamin, obesity and hair loss genes received from a genetic analysis company;
a self-examination information input unit (300) for inputting, through Internet (I) or Wi-Fi (W1), self-examination percentage data obtained by checking, at a user's PC (100) and a smartphone (200), each of 33 questionnaire items in a skin and metabolism self-examination list and converted into percentages using the Likert scale of measurement;
a data storage unit (410B) constituting a genetic analysis server (410) in a cloud server (400) for storing the genetic percentage data and self-examination information percentage data obtained by the self-examination information input unit (300) and an examination analysis processing unit (410A) for a comparative analysis of the stored input data; and
a data relay (420) in the cloud server (400) for inputting, through Wi-Fi (W2), moisture and oil data and pigment, pore and wrinkle image data obtained by an IoT skin measurement terminal (500),
wherein the skin measurement data is transmitted by the data relay (420) in the cloud server (400) to the user's PC (100) or smartphone (200) through Internet (I) or Wi-Fi (W1) to identify an authentication number of the corresponding IoT skin measurement terminal (500) for authentication processing, and once authentication is confirmed, the skin measurement data of the corresponding IoT skin measurement terminal (500) is transmitted to an image analysis server (430) in the cloud server (400) by operation of the user's PC (100) or smartphone (200) to compare, by a skin measurement data analysis unit (430B), a reference database (430A) constituting the image analysis server (430) and the skin measurement data input, such that the produced percentage data of each of oil, pigment, pore and wrinkle is transmitted to the genetic analysis server (410), wherein the genetic analysis server (410) compares the genetic percentage data obtained by the genetic analysis database (10), the self-examination percentage data obtained by the self-examination information input unit (300) and the skin measurement percentage data obtained by the image analysis server (430) using the examination analysis processing unit (410A), and when the self-examination percentage data obtained by the self-examination information input unit (300) and the genetic percentage data obtained by the genetic analysis database (10) are input, an average percentage data value of each genetic item is subtracted from an average percentage data value of each self-examination item to determine a smallest percentage data value of the percentage data values of the corresponding items as a corresponding code and when the self-examination percentage data and skin measurement percentage data obtained by the image analysis server (430) are input together with the genetic percentage data to the analysis server (410), the percentage data of the self-examination item is replaced with the percentage data of the corresponding skin measurement item, and an average percentage data value of each genetic item is subtracted from an average percentage data value of each corresponding self-examination item to determine a smallest percentage data value of the percentage data values of the corresponding items as a corresponding examination code, such that the determined examination code is transmitted to the user's PC (100) and smartphone (200), thereby providing a cosmetic suitable for skin symptoms, cause, prevention and regeneration according to the transmitted examination code.

2. The system of claim 1, wherein the IoT skin measurement terminal (500) comprises moisture and oil sensing units (510)(520), a skin photographing camera (530) and an LED light unit (540), wherein the data measured by the moisture and oil sensing units (510)(520) and skin photographing camera (530) is compared with the reference data of the reference database (430A) having a plurality of image data and converted into percentage data by the skin measurement data analysis unit (430B), wherein the IoT skin measurement terminal (500) provides a web-based examination solution which exchanges a protocol with the cloud server (400) and the user's PC (100) and smartphone (200).

3. The system of claim 1, wherein skin types are classified into dry, sensitive, oily, pigment and wrinkle and further classified into 10 subdivided skin items to classify an individual's skin characteristic, wherein the average percentage data values of the genetic items are subtracted from the average percentage data values of the corresponding self-examination items, and a smallest value thereof matches with 10 subdivided skin types classified as the subdivided items, thereby determining an examination code with English capital letters.

4. The system of claim 1, wherein the summed-up percentages of gene items corresponding to dry in the genetic item percentage data input to the genetic analysis server (410) are converted into an average value at the examination analysis processing unit (410A), the summed-up percentages of dry moisture item corresponding to dry of the skin symptom type obtained by checking the self-examination questionnaire item at the user's PC or smartphone and converted using the Likert scale are converted into an average value at the examination analysis processing unit (410A) and at the same time the summed-up percentages of dry oily item corresponding to dry obtained by checking the self-examination questionnaire item and converted using the Likert scale are converted into an average value at the examination analysis processing unit (410A), dry moisture type percentage data is obtained by subtracting the genetic analysis average value corresponding to dry from the dry moisture type average value by the self-examination (self-examination analysis average value−genetic analysis average value), dry oily type percentage data is obtained by subtracting the genetic analysis average value from the dry oily type average value (self-examination analysis average value−genetic analysis average value), then a smaller value between the dry moisture type percentage data and dry oily type percentage data is produced at the examination analysis processing unit (410A), in the same manner as above, the percentage data for each of 8 subdivided skin items corresponding to sensitive, oily, pigment and wrinkle skin types is summed up sequentially to produce each average value by the examination analysis processing unit (410A), to produce and provide, as an examination code, the item having a smallest value of the 10 skin types of dry, sensitive, oily, pigment and wrinkle.

5. A method for analyzing and providing personalized cosmetic information, comprising:
a step (S1) of receiving genetic information which is receiving, from a genetic analysis DB, 28 pieces of skin-related percentage data received from a genetic analysis company;
a step (S2) of transmitting genetic information which is transmitting the genetic percentage data of the genetic analysis DB to a genetic analysis server in a cloud server through HTTP REST API;
a step (S3) of transmitting self-examination skin condition information which is transmitting self-examination skin region percentage data obtained from self-examination questionnaire items using the Likert scale to the genetic analysis server in the cloud server through a self-examination information input unit;
a step (S4) of measuring skin which is measuring moisture, oil, pore, pigment and wrinkle conditions through a skin measurement algorithm of an IoT skin measurement terminal;
a step (S5) of transmitting the skin measurement data of the IoT skin measurement terminal wherein checked items in the self-examination questionnaire items are replaced with moisture and oil data and pore, pigment and wrinkle photographed image data measured by the IoT skin measurement terminal to be input, the skin measurement data is transmitted by a data relay in the cloud server to a user's PC or smartphone through Internet or Wi-Fi to identify an authentication number of the corresponding IoT skin measurement terminal for authentication processing, and once authentication is confirmed, the skin measurement data of the corresponding IoT skin measurement terminal is transmitted to an image analysis server in the cloud server through a second skin measurement data processing unit by operation of the user's PC or smartphone to compare, by a skin measurement data analysis unit, a reference database constituting the image analysis server and the skin measurement data input, such that the produced percentage data of each of moisture, oil, pigment, pore and wrinkle is transmitted to the genetic analysis server;
a step (S6) wherein the sum of percentage data of skin genetic marker items corresponding to dry in the genetic item percentage data input to the genetic analysis server is converted into an average value at an examination analysis processing unit, the sum of percentages of the percentage data of dry moisture type question 1 and the percentage data of dry moisture type question 2 obtained by checking the self-examination questionnaire item and converted using the Likert scale and the moisture and oil data and pore, pigment and wrinkle photographed image data measured by the IoT skin measurement terminal corresponding thereto are converted into an average value at the examination analysis processing unit, and at the same time the sum of percentages of the percentage data of dry oily type question 1 and the percentage data of dry oily type question 2 obtained by checking the self-examination questionnaire item and converted using the Likert scale is converted into an average value at the examination analysis processing unit, then the average value of the sum of the moisture and oil is set as an average value for large-category skin type I, the genetic analysis average value corresponding to dry is subtracted therefrom (self-examination analysis average value−genetic analysis average value) to obtain dry percentage data, and a smaller value between the moisture and oil items in the self-examination is determined as a dry moisture type examination code of subdivided skin type II;
a step (S7)(S7′)(S7″)(S7′″) wherein an average value of percentage data of each genetic item of sensitive, oily, pigment, and wrinkle, an average value of percentage of each sensitive, oily, pigment and wrinkle checked in the self-examination items and the moisture and oil data and pore, pigment and wrinkle photographed image data measured by the IoT skin measurement terminal corresponding thereto, which are input to the genetic analysis server of the cloud server, are compared and analyzed at the examination analysis processing unit of the genetic analysis server as in the step (S6) for the subdivided skin type II items (8 items) for each of sensitive, oily, pigment and wrinkle;
a step (S8) of determining a smallest value (%) of the subdivided skin type II items (10 items) corresponding to the large-category skin type I (5 items) compared and analyzed in the step of subtraction processing (S6)(S7)˜(S7′″);
a step (S9) of analysis processing which is determining the code produced in the step (S8) as an examination code according to a prescription for skin symptoms;
a step (S10) of determining in the order of dry<sensitive<oily<pigment<wrinkle when the result values of the examination codes determined in the analysis processing step (S9) are identical;
a step (S11) of transmitting the determined examination code to the user's PC or smartphone; and
a step (S12) of providing a cosmetic that can solve skin problems based on the determined examination code.
